(19) United States
(12) Patent Application Publication
(10) Pub. No.: US 2022/0290539 A1
(43) Pub. Date: Sep. 15, 2022

(54) OIL-IN-WATER ANALYZER FOR PRODUCED WATER

(71) Applicant: Saudi Arabian Oil Company, Dhahran (SA)

(72) Inventor: Abdulrahman Ali Alshehri, Udhailiyah (SA)

(21) Appl. No.: 17/197,572

(22) Filed: Mar. 10, 2021

(51) Int. Cl. E21B 43/16; E21B 47/047; G06N 7/00

(52) U.S. Cl. CPC E21B 43/16; E21B 47/047; G06N 7/005; E21B 2200/22

(57) ABSTRACT

The subject matter of this specification can be embodied in, among other things, a method that includes receiving a collection of process parameters associated with disposal of water from a hydrocarbon extraction process, the collection of process parameters including a demulsifier flowrate, a hydrocarbon temperature, a first high pressure production trap (HPPT) interface level, a second HPPT interface level, a produced water rate, a dehydrator interface level, a desalter interface level, a tie line production rate, a water-oil separator oil level, and a water-in-oil separator water level, determining an oil-in-water (OIW) concentration in hydrocarbons from the hydrocarbon extraction process using the collection of process parameters according to a first order linear regression model, comparing the determined OIW concentration to a threshold concentration, and adjusting at least one of the collection of process parameters associated with the hydrocarbon extraction process based on the comparing.

### TECHNICAL FIELD

The present disclosure generally relates to the production of crude oil, more particularly, to the determining the concentration of oil in disposal water in crude oil.

### BACKGROUND

The production of crude oil may involve a number of processes engineered to produce crude oil to specific quality specifications. For example, extracted oil can contain water that needs to be removed and appropriately disposed of. Thus, the production of crude oil may include processes and techniques to remove water from crude oil. However, disposal water often includes residual oil, also called oil-in-water (OIW). In some instances, the OIW content level in produced water may be manually monitored by sampling water from the plant disposal system and at time intervals. The sampled foundation water (e.g., produced water) samples are then sent to a central lab for analysis and result. The available technologies to provide a continuous reading of the oil in water content level in the disposal system are full of complexity and will inquire initial installation cost, and periodic maintenance cost.

The results of the sampling may provide the amount of OIW. However, such sampling may only be used to reactively change process parameters to improve produced water quality, maximized the plant operating efficiency, and maintain disposal wells injectivities. Moreover, such manual sampling may be tedious and time-consuming and only detects deterioration in OIW quality after it has occurred in the time interval between samplings.

### SUMMARY

In general, the present disclosure describes techniques used to determine the concentration of oil in disposal water in crude oil.

In a general aspect, a computer-implemented method includes receiving a collection of process parameters associated with disposal of water from a hydrocarbon extraction process, the collection of process parameters comprising a demulsifier flowrate, a hydrocarbon temperature, a first high pressure production trap (HPPT) interface level, a second HPPT interface level, a produced water rate, a dehydrator interface level, a desalter interface level, a tie line production rate, a water-oil separator oil level, and a water-in-oil separator water level, determining an oil-in-water (OIW) concentration in hydrocarbons from the hydrocarbon extraction process using the collection of process parameters according to a first order linear regression model, comparing the determined OIW concentration to a threshold concentration, and adjusting at least one of the collection of process parameters associated with the hydrocarbon extraction process based on the comparing.

Various implementations can include some, all, or none of the following features. The method can also include providing a notification based on the comparing. The method can also include receiving a sample of hydrocarbons output from the hydrocarbon extraction process, and comparing the determined OIW concentration to an OIW concentration determined from the received sample. Determining the OIW concentration in hydrocarbons from the hydrocarbon extraction process using the collection of process parameters can include determining the OIW concentration according to the following: OIW=β₀+β₁A+β₂B+β₃C+β₄D+β₅E+β₆F+β₇G+β₈H+β₉I+β₁₀J+ε, where OIW can be the oil-in-water concentration, A can be the demulsifier flowrate, B can be the hydrocarbon temperature, C can be the first HPPT interface level, D can be the second HPPT interface level, E can be the produced water rate, F can be the dehydrator interface level, G can be the desalter interface level, H can be the tie line production rate, I can be the water-oil separator oil level, J can be the water-in-oil separator water level, c is a random error term, and β₀, β₁, β₂, β₃, β₄, β₅, and β₆ are factor effects. Receiving a collection of process parameters can include periodically receiving the collection of process parameters, and determining an OIW concentration can include periodically determining the OIW concentration.

In another example embodiment, a non-transitory, computer-readable medium storing one or more instructions is executable by a computer system to perform operations including receiving a collection of process parameters associated with disposal of water from a hydrocarbon extraction process, the collection of process parameters including a demulsifier flowrate, a hydrocarbon temperature, a first high pressure production trap (HPPT) interface level, a second HPPT interface level, a produced water rate, a dehydrator interface level, a desalter interface level, a tie line production rate, a water-oil separator oil level, and a water-in-oil separator water level, determining an oil-in-water (OIW) concentration in hydrocarbons from the hydrocarbon extraction process using the collection of process parameters according to a first order linear regression model, comparing the determined OIW concentration to a threshold concentration, and adjusting at least one of the collection of process parameters associated with the hydrocarbon extraction process based on the comparing.

Various embodiments can include some, all, or none of the following features. The operations can also include providing a notification based on the comparing. The operations can also include receiving a sample of hydrocarbons output from the hydrocarbon extraction process, and comparing the determined OIW concentration to an OIW concentration determined from the received sample. Determining the OIW concentration in hydrocarbons from the hydrocarbon extraction process using the collection of process parameters can include determining the OIW concentration according to the following: OIW=β₀+β₁A+β₂B+β₃C+β₄D+β₅E+β₆F+β₇G+β₈H+β₉I+β₁₀J+ε, where OIW can be the oil-in-water concentration, A can be the demulsifier flowrate, B can be the hydrocarbon temperature, C can be the first HPPT interface level, D can be the second HPPT interface level, E can be the produced water rate, F can be the first dehydrator interface level, G can be the second dehydrator interface level, H can be the tie line production rate, I can be the water-oil separator oil level, J can be the water-in-oil separator water level, ε is a random error term, and β₀, β₁, β₂, β₃, β₄, β₅, and β₆ are factor effects. Receiving a collection of process parameters can also include periodically receiving the collection of process parameters, and determining an OIW concentration can include periodically determining the OIW concentration.

In another example aspect, a computer-implemented system includes one or more processors, and a non-transitory computer-readable storage medium coupled to the one or more processors and storing programming instructions for execution by the one or more processors, the programming instructions instructing the one or more processors to perform operations including receiving a collection of process parameters associated with disposal of water from a hydrocarbon extraction process, the collection of process parameters including a demulsifier flowrate, a hydrocarbon temperature, a first high pressure production trap (HPPT) interface level, a second HPPT interface level, a produced water rate, a dehydrator interface level, a desalter interface level, a tie line production rate, a water-oil separator oil level, and a water-in-oil separator water level, determining an oil-in-water (OIW) concentration in hydrocarbons from the hydrocarbon extraction process using the collection of process parameters according to a first order linear regression model, comparing the determined OIW concentration to a threshold concentration, and adjusting at least one of the collection of process parameters associated with the hydrocarbon extraction process based on the comparing.

Various embodiments can include some, all, or none of the following features. The operations can also include providing a notification based on the comparing. The operations can also include receiving a sample of hydrocarbons output from the hydrocarbon extraction process, and comparing the determined OIW concentration to an OIW concentration determined from the received sample. Determining the OIW concentration in hydrocarbons from the hydrocarbon extraction process using the collection of process parameters can include determining the OIW concentration according to the following: OIW=β₀+β₁A+β₂B+β₃C+β₄D+β₅E+β₆F+β₇G+β₈H+β₉I+β₁₀J+ε, where OIW can be the oil-in-water concentration, A can be the demulsifier flowrate, B can be the hydrocarbon temperature, C can be the first HPPT interface level, D can be the second HPPT interface level, E can be the produced water rate, F can be the first dehydrator interface level, G can be the second dehydrator interface level, H can be the tie line production rate, I can be the water-oil separator oil level, J can be the water-in-oil separator water level, ε is a random error term, and β₀, β₁, β₂, β₃, β₄, β₅, and β₆ are factor effects. Receiving a collection of process parameters can include periodically receiving the collection of process parameters, and determining an OIW concentration can include periodically determining the OIW concentration.

The subject matter described in this specification can be implemented in particular implementations, so as to realize one or more of the following advantages. First, a system can estimate or predict the concentration of oil in water without physical sampling or traditional lab analysis. Second, the system can estimate or predict the concentration of oil in water in near real time. Third, the system can control a gas and oil separation process, based on the estimated or predicted concentration of oil in water, to preserve a disposal system's piping and/or a wellhead's integrity from scale and corrosion growth. Fourth, the system can maintain disposal well injectivity performance. Fifth, the system can prevent disposal well plugging and high bottom-hole pressure.

The details of one or more implementations are set forth in the accompanying drawings and the description below. Other features and advantages will be apparent from the description and drawings, and from the claims.

### DESCRIPTION OF DRAWINGS

FIG. 1 depicts an example plant information system in communication with an example water separation process.

FIG. 2 is a block diagram that shows an example collection of GOSP parameters that can be used to determine the example oil-in-water concentration model.

FIG. 3 shows an example table that represents a sampling methodology that can be used to collect data.

FIG. 4 is a graph of a comparison between an example collection of measured oil-in-water concentration values and an example collection of predicted oil-in-water concentration values.

FIG. 5 is a flow diagram of an example process 500 for determining an oil-in-water concentration.

FIG. 6 is a block diagram of an example computer system.

### DETAILED DESCRIPTION

Embodiments of the disclosure generally relate to an oil-in-water (OIW) analyzer for crude oil. The OIW analyzer includes an OIW concentration model that determines an OIW concentration from hydrocarbon extraction and water separation process parameters. The OIW concentration model may be a first order continuous variables model and may be determined using a regression analysis and sample data obtained from a water separation process, such as from an oil-gas separation plant. The OIW analyzer may compare the oil concentration in the produced water to a threshold concentration to determine if the oil concentration in the produced water exceeds the threshold concentration and may perform or initiate actions based on the comparison.

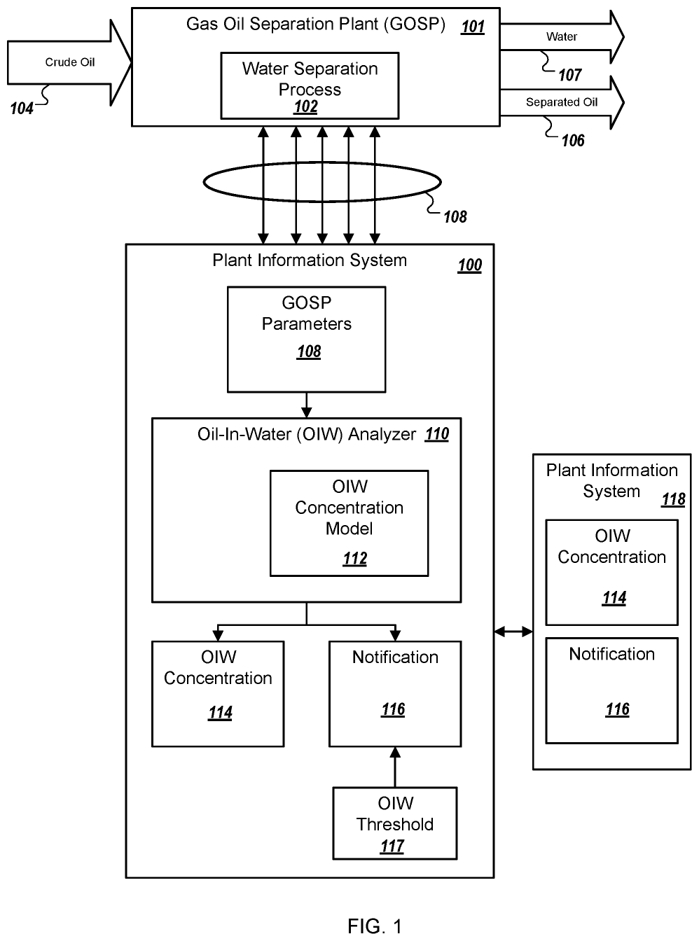

In some embodiments, an OIW analyzer having an OIW concentration model may be implemented in a plant information system. FIG. 1 depicts an example plant information (PI) system 100 in communication with an example gas and oil separation plant (GOSP) 101. The GOSP 101 is configured to perform an example water separation process 102 (e.g., an oil dehydration process) in accordance with an embodiment of the disclosure. In some embodiments, the plant information system 100 may interface with a distributed control system (not shown) that controls various components that control and monitor the water separation process 102. It should be appreciated that, in other embodiments, an OIW analyzer having an OIW concentration model may be implemented in the distributed control system instead of the plant information system 100.

The GOSP 101 may receive crude oil 104 having a relatively high water content and output a dehydrated crude oil 106 and a disposal water 107. The water separation process 102 may include any suitable water/oil separation processes and techniques, such as single stage dehydrators and multi-stage dehydrators. As shown in FIG. 1, the plant information system 100 may receive GOSP parameters 108 from the water separation process 102. For example, the GOSP parameters 108 may be measured using suitable components arranged at various locations around the water separation process 102. Such components may include sensors such as flowmeters, temperature sensors (e.g., thermometers or thermocouples), voltage sensors, and combinations of these and any other appropriate sensor or input that can be used with an oil extraction or water separation process. Such components may be a part of the plant information system 100 or a distributed control system, or may be communication with the plant information system 100 or distributed control system via a suitable communication network.

In some embodiments, the GOSP parameters include a demulsifier flowrate, a hydrocarbon (crude oil) temperature, a first high pressure production trap (HPPT) interface level, a second HPPT interface level, a produced water rate, a dehydrator interface level, an interface level, a dehydrator transformer grid voltage, a desalter transformer grid voltage, a tie line production rate, a water-oil separator oil level, and a water-in-oil separator water level. The OIW analyzer 110 may determine the OIW concentration 114 without interruption of the water separation process 102 without physically sampling the crude oil 104, and may provide faster determinations of the OIW concentration 114 as compared to laboratory analysis of crude oil samples. Additionally, the OIW analyzer 110 may provide for predictions of oil concentration and maintenance of a desired oil concentration in the disposal water system 107.

As described above, the GOSP parameters 108 may be provided to an OIW analyzer 110. The OIW analyzer 110 may implement an OIW concentration model 112 determined in accordance with the techniques described in the disclosure. In general, the OIW concentration model 112 is determined using mathematical regression modelling based on measured or observed operational parameters under various operational conditions and analytically determined OIW concentrations that result from those conditions.

The OIW analyzer 110 may receive the GOSP parameters 108 and determine an OIW concentration 114 for the disposal water 107 output from the water separation process 102. As described above, in some embodiments the OIW analyzer 110 may obtain the GOSP parameters 108 and determine the OIW concentration 114 periodically or upon request, such as from an operator interfacing with the plant information system 100. In some embodiments, as described above, the OIW analyzer 110 may provide a notification 116 if the OIW concentration exceeds an OIW threshold concentration 117. For example, in some embodiments the notification may include the activation of an alarm of the plant information system 100. In another example, if the determined OIW concentration 114 is below the OIW threshold concentration 117, the OIW analyzer 110 may continue monitoring of the GOSP parameters 108 and the OIW concentration 114. If the determined OIW concentration 114 is above the OIW threshold concentration 117, the notification 116, such as a notification in a plant information (PI) system, may be provided.

In some embodiments, if the determined OIW concentration 114 is above the OIW threshold concentration 117, the GOSP parameters 108 may be adjusted, such as via a distributed control system (DCS). For example, the OIW analyzer 110 may be used to create a reference operating point for the plant separation process parameters time period or a number of samples. In another example, one or more parameters associated with separation process can be adjusted if the oil in water concentration exceeds a predetermined concentration limit. In yet another example, a recycling mechanism of the disposal system can be engaged to recycle water output back through the system for further processing and separation if the oil in water concentration exceeds the predetermined concentration limit.

In the illustrated example, the OIW analyzer 110 is implemented in the plant information system 100, which is in communication with the water separation process 102. In some embodiments, the plant information system 100 may receive the water-oil separation process parameters 108 from the water separation process 102, and the OIW analyzer 110 may determine the OIW concentration 114 in the disposal water 107 output from the water separation process 102 using the OIW concentration model 112. In such embodiments, in response to a determined OIW concentration 114, the plant information system 100 may provide the OIW concentration 114, the notification, or both to a plant information client 118 in communication with the plant information system 100. In some embodiments, the plant information client 118 may include a display that displays an alert in response to the notification 116 or other data received from the plant information system 100.

In some embodiments, the plant information client 118 may provide plant information to an operator and enable monitoring of various process parameters. In such embodiments, the plant information system 100 may provide information to the plant information client 118 about the water separation process 102. For example, the plant information system 100 may provide the OIW concentration 114 determined by the OIW analyzer 110 to the plant information client 118. Alternatively or additionally, in some embodiments the plant information system 100 may provide the notification 116 to the plant information client 118. For example, in some embodiments the notification 116 may include an alarm displayed on the plant information client 118 that indicates that the OIW concentration 114 has exceeded a threshold concentration. The notification 116 may, for example, enable an operator to take actions regarding any changes in OIW concentration provided via the plant information client 118.

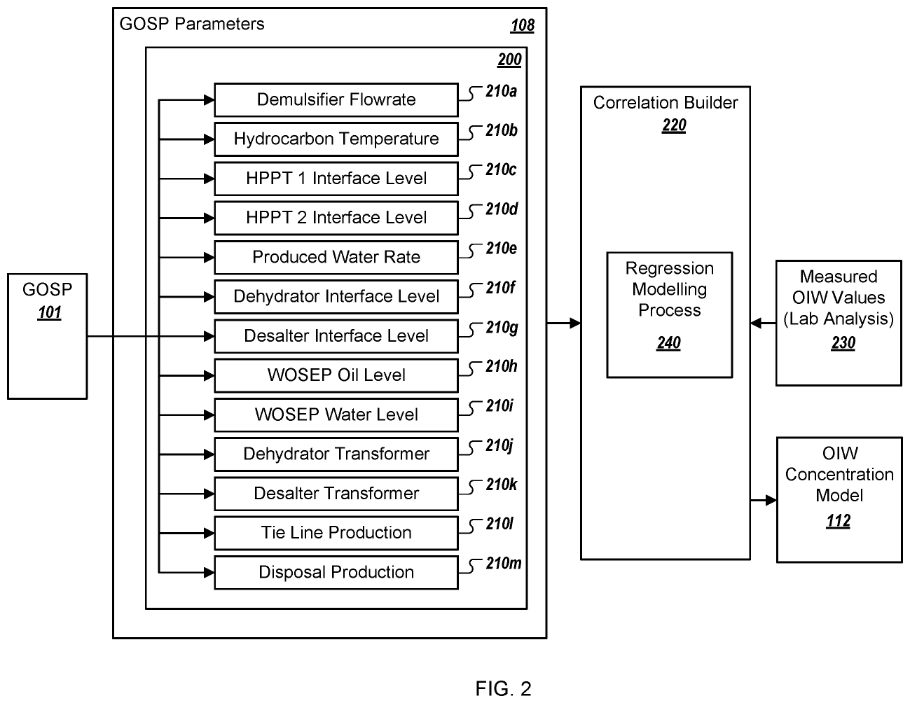

FIG. 2 is a block diagram that shows an example collection of GOSP parameters 200 that can be used to determine the example OIW concentration model 112 of FIG. 1. In some implementations, the GOSP parameters 200 can be some or all of the example GOSP parameters 108.

The GOSP parameters 200 include:

a demulsifier flowrate 210a (e.g., in gallons per day);

a hydrocarbon temperature 210b (e.g., in degrees Fahrenheit);

a first high pressure production trap (HPPT) interface level 210c (e.g., as a percentage);

a second HPPT interface level 210d (e.g., as a percentage);

a produced water rate 210e (e.g., in gallons per day);

a dehydrator interface level 210f (e.g., as a percentage);

a desalter vessel interface level 210g (e.g., as a percentage);

a water-in-oil separator (WOSEP) oil level 210h (e.g., as a percentage);

a water-in-oil separator water level 210i (e.g., as a percentage);

a dehydrator transformer level 210j (e.g., on or off);

a desalter transformer level 210k (e.g., on or off);

a tie line production rate 210l (e.g., in thousands of barrels per day); and

a disposal water production rate 210m (e.g., gallons per day).

The GOSP parameters 200 are provided to a correlation builder 220 as a collection of independent variables for use in a regression modelling process 240. A collection of measured OIW values 230 are provided to the correlation builder 220 as a collection of dependent variables for use by the correlation builder 220 in the regression modelling process 240. For example, the GOSP parameters 200 for a first set of operational conditions (e.g., temperature, extraction rate) can result in a first amount of OIW in disposal water, while the GOSP parameters 200 for a different set of operational conditions can result in a different amount of OIW in disposal water. The regression modelling process 240 implements a set of processing parameter values to determine the relation between the dependent variable and one or more of independent variables.

The OIW concentration model 112 is a mathematical model that can be used to predict, estimate, or otherwise determine the OIW concentration 114 from different OIW and GOSP parameters, such as the example GOSP parameters 108. In some embodiments, the OIW concentration model 112 can be a first order continuous variables model generated from correlations between the GOSP parameters 108 and the corresponding OIW concentrations, as observed in the measured OIW values 230. In some implementations, the correlation builder 220 can be part of the example plant information system 100 of FIG. 1 (e.g., part of the OIW analyzer 110), or the correlation builder 220 can be implemented in a separate system that is configured to determine the OIW concentration model 112 and provide it to the OIW analyzer 110 for operational use.

In some embodiments, the first order continuous variables model used in the correlation builder 220 may be represented as Equation 1:

OIW=β₀+β₁A+β₂B+β₃C+β₄D+β₅E+β₆F+β₇G+β₈H+β₉I+β₁₀J+ε (Equation 1)

where:

OIW is the oil-in-water concentration in parts per million (PPM);

A is the demulsifier flowrate in gallons per day;

B is the hydrocarbon temperature in degrees Fahrenheit;

C is the first HPPT interface level as a percentage;

D is the second HPPT interface level as a percentage;

E is the produced water rate in thousands of barrels per day;

F is the dehydrator interface level as a percentage;

G is the desalter interface level as a percentage;

H is the tie line production rate in thousands of barrels per day;

I is the water-in-oil separator oil level as a percentage;

J is the water-in-oil separator water level as a percentage;

ε is a random error term;

and β₀, β₁, β₂, β₃, β₄, β₅, and β₆ are factor effect values (e.g., correlation factors, factor effects).

Since the example factor effect values β₀, β₁, β₂, β₃, β₄, β₅, and β₆ of Equation 1 can be determined through regression modelling, their accuracy may be dependent upon the quality and diversity of available data that can be used as the GOSP parameters 108 and the measured OIW values 230. In some implementations, a wide spectrum of operational data may be collected or otherwise obtained and used to improve the quality of the factor effect values, which in turn can improve the ability of Equation 1 to estimate or predict the OIW concentration 114 under various operational conditions.

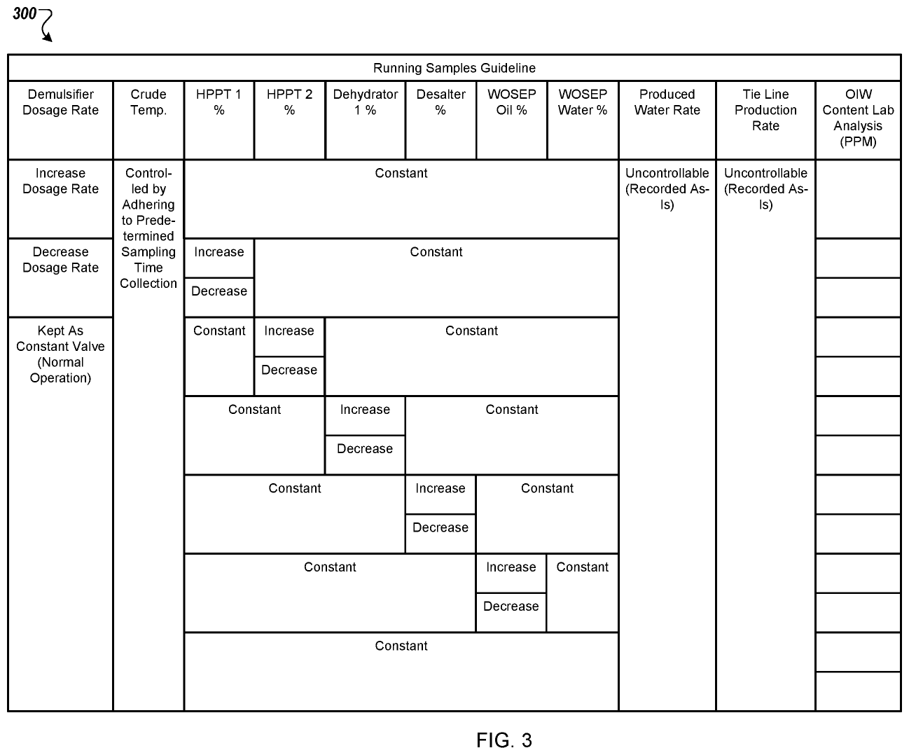

In some implementations, the GOSP parameters 108 and the measured OIW values 230 can be obtained through a predetermined process. FIG. 3 shows an example table 300 that represents a sampling methodology that can be used to collect data (e.g., the GOSP parameters 108, and then observe the oil in water values (e.g., measured OIW values 230) for a predetermined process setting.

In this method, controllable independent process parameters (e.g., demulsifier, production volumes, HPPT interface level percentages, dehydrator and desalter interface level percentages, WOSEP oil & water level percentages) can be tested individually while holding all others substantially unchanged (e.g., to act as constant or dependent values while changing one or few controlled values). The constant parameters' values can be predetermined values, such as values specified by GOSP operation instruction manual (OIM) setpoints. In some implementations, the effect of temperature on the disposal water OIW content level can be tested by changing the timing of sample collection (e.g., sampling in the morning, afternoon, or at night).

In addition, uncontrollable parameters such as the plant water disposal rate and plant oil production rate can be recorded as it is during the sampling collection. Desalter and dehydrator transformer grid voltages can be observed as digital values represented as either on or off (e.g., 0 or 1). In some implementations, the generated regression model can be regarded as invalid if the transformers' grids are off (0), which can imply that the plant produced crude oil and disposal water may be out of specification.

At each test, the above mentioned process parameters can be recorded along with OIW values that can be determined using a conventional testing method (e.g., in a central testing lab) to build up the foundation for regression modeling. Finally, the test is decided by p-values obtained by the regression analysis. For example, MEGASTAT software can be used as an add-in to EXCEL spreadsheet software and used to perform regression analysis based on spreadsheet data). In some implementations, if the p-value of any process parameter less than 0.05, then the parameter can be considered to be mathematically significant, otherwise the process parameter value can be rejected and the coefficient (β0-β10) in Equation (1) can be set to zero.

Currently, GOSPs typically measure the oil in water content for the disposal system on a substantially continuous basis, utilizing conventional methods (e.g., physically collecting samples to be analyzed by a central laboratory). Thus, a massive historical data of oil in water values in the disposal system may be accessible for each operating facility. In some implementations, the GOSP parameters 108 and the measured OIW values 230 can be obtained from historical operational process data. For example, during oil extraction processes, the example GOSP 101, the plant information system 100, the plant information client 118, or human operators may measure and record (e.g., automatically or manually) at least some of the GOSP parameters 108 and the measured OIW values 230 as part of the normal extraction operations. Such recorded data could be processed to extract the GOSP parameters 108 and the measured OIW values 230.

In this method (for example, to avoid inconvenience to the plant console operator), historical OIW analysis (e.g., performed by a central lab) can be used instead of running a new testing samples. Historical data generally includes the exact date and time recorded for when the samples were collected. The recorded dates and times can be used to fetch the independent process parameters from a database (e.g., of the plant information system 100). In examples where a mathematically sufficient and diverse amount of operational data exists, such data can be used in the regression modeling process, reducing or eliminating the need to collect the information illustrated by FIG. 3.

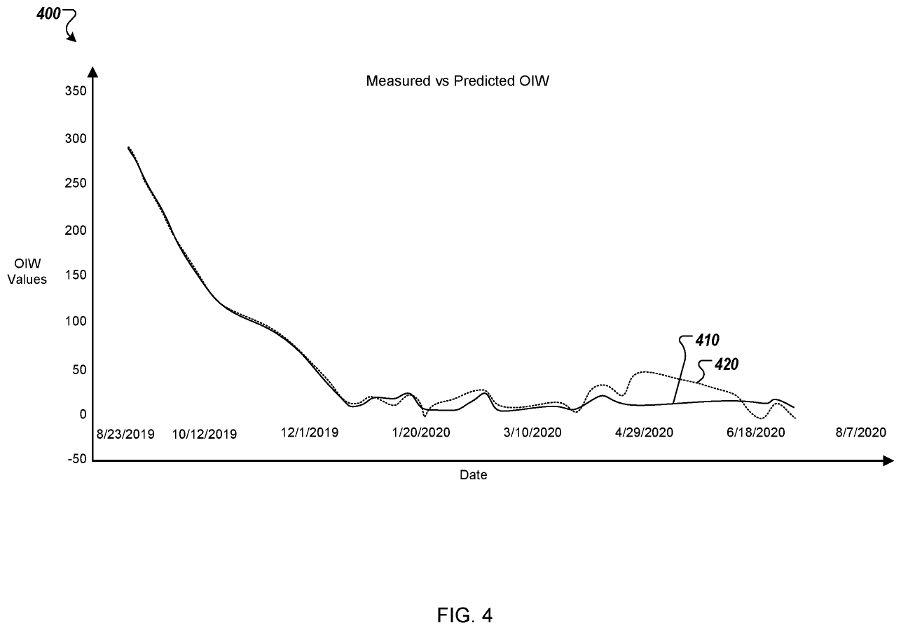

FIG. 4 is a graph 400 of a comparison between an example collection of measured OIW concentration values 410 and an example collection of predicted OIW concentration values 420. The illustrated example is included to demonstrate embodiments of the disclosure. It should be appreciated by those of skill in the art that the techniques and compositions disclosed in the example which follows represents techniques and compositions discovered by the inventors to function well in the practice of the disclosure, and thus can be considered to constitute modes for its practice. However, those of skill in the art should, in light of the present disclosure, appreciate that many changes can be made in the specific embodiments which are disclosed and still obtain a like or a similar result without departing from the spirit and scope of the disclosure.

A collection of historical data was implemented at a selected GOSP for a first piloted trail. A total of 30 historical samples were used to generate and test the hypotheses of the first order regression modeling. For each sample, the process parameters were fetched from a plant information distributed control system, and the particular dates and times of when the samples were collected were recorded. The data collection process was designed to identify a variety of OIW values in order to give the model a bigger scale (e.g., range) for value anticipation (e.g., highest ˜290 PPM, lowest ˜5 PPM).

After getting a variety of samples, the data were modeled using MEGASTAT add-in tool for EXCEL software to perform the regression analysis. Accordingly, beta (e.g., β₀, β₁, β₂ . . . ) coefficients in Equation (1) were calculated which will produce the model. The generated algorithm was verified by comparing the OIW lab analysis feedback (e.g., the collection 410) versus the newly generated model (e.g., the collection 420) to check the accuracy. The result was encouraging in predicting the impact of the plant process parameters upon the disposal water quality.

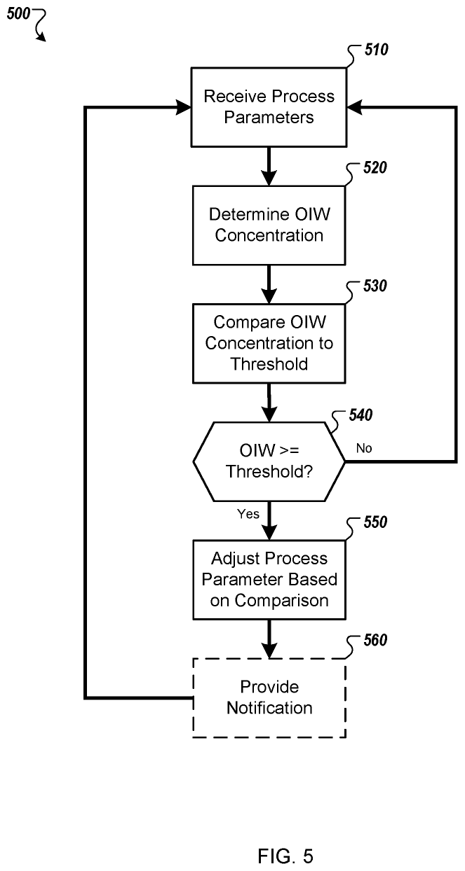

FIG. 5 is a flow diagram of an example process 500 for determining an OIW concentration. In some implementations, the process 500 can be performed by some or all of the example plant information system 100 (e.g., the OIW analyzer 110) of FIG. 1, the example plant information client 118, the example GOSP 101, or combinations of these and any other appropriate system that can be used to determine OIW concentrations.

At 510, a collection of process parameters associated with disposal of water from a hydrocarbon extraction process is received. The collection of process parameters includes a demulsifier flowrate, a hydrocarbon temperature, a first high pressure production trap (HPPT) interface level, a second HPPT interface level, a produced water rate, a first dehydrator interface level, a second dehydrator interface level, a tie line production rate, a water-oil separator oil level, and a water-in-oil separator water level. For example, the plant information system 100 can receive the GOSP parameters from the GOSP 101.

At 520, an oil-in-water (OIW) concentration in hydrocarbons from the hydrocarbon extraction process is determined using the collection of parameters according to a first order linear regression model. For example, the OIW analyzer 110 can determine the OIW concentration 114 in the disposal water 107 based on the GOSP parameters 108 and the OIW concentration model 112.

At 530, the determined OIW concentration is compared to a threshold concentration. For example, the plant information system 100 can compare the OIW threshold concentration 117 to the OIW concentration 114.

At 540, a determination is made. If the OIW concentration is less than the threshold concentration, then the process 500 continues at 510. In the OIW concentration is greater than or equal to the threshold concentration, then the process 500 continues at 550.

At 550, at least one of the collection of parameters associated with the hydrocarbon extraction process is adjusted based on the comparing. For example, the plant information system 100 can determine that the OIW concentration 114 has exceeded the OIW threshold concentration 117, and respond by identifying the offset (e.g., determining what went wrong) in the process parameter based on the reference operating point identified earlier. Based on the identification, the plant information system 100 may provide a corrective process setting based on the reference operating point. In another example, the plant information system 100 may respond by triggering an alarm process, by sharing current OIW concentration, the offset in process, and a corrective setting, or by providing a combination of these and other appropriate forms of notifications and information.

At 560, an optional notification can be provided based on the comparing. For example, the plant information system 100 or the plant information client 118 can provide an alert (e.g., display a message on a display, send an email or text message to appropriate personnel), trigger an alarm (e.g., a siren, a buzzer, a signal light), or provide any other appropriate form of communication that can convey information about or draw attention to the determined high OIW concentration level. The process 500 then continues at 510.

In some implementations, the process 500 can include receiving a sample of hydrocarbons output from the hydrocarbon extraction process, and comparing the determined OIW concentration to an OIW concentration determined from the received sample. For example, a sample of the disposal water 107 can be sent to a laboratory for analysis to determine a level of hydrocarbons that are present in the disposal water 107 (e.g., an OIW concentration). The OIW concentration determined by the lab analysis can then be compared to the OIW concentration 114 determined by the OIW analyzer 110. In some implementations, the lab-determined OIW concentration can be used to verify or validate the OIW concentration model 112. In some implementations, the lab-determined OIW concentration can be used to refine the OIW concentration model 112 further.

In some implementations, determining the OIW concentration in hydrocarbons from the hydrocarbon extraction process using the collection of process parameters can include determining the OIW concentration according to Equation 1.
In some implementations, receiving a collection of process parameters can include periodically receiving the collection of parameters, and determining an OIW concentration can include periodically determining the OIW concentration. For example, the process 500 may be performed on a predetermined interval (e.g., once per minute, once per hour, once per day). In another example, the process 500 may be performed in response to operator input (e.g., on demand). In another example, the process 500 may be performed in response to changes in the GOSP parameters 108 (e.g., the process 500 may be performed every time the hydrocarbon temperature (e.g., parameter “B” of Equation 1) changes by more than a predetermined amount (e.g., one degree, three degrees), or when a high pressure production trap (HPPT) interface level changes by more than a predetermined percentage (e.g., 2%, 5%).

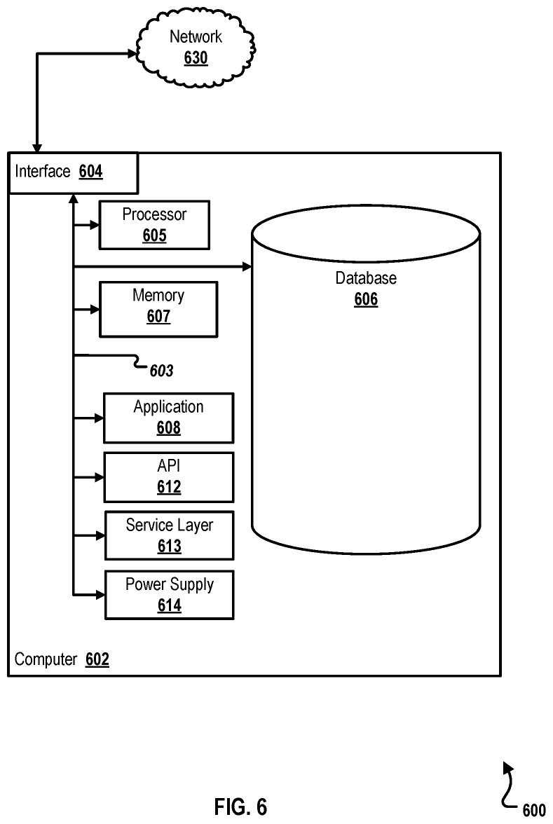

FIG. 6 is a block diagram of an example computer system 600 used to provide computational functionalities associated with described algorithms, methods, functions, processes, flows, and procedures described in the present disclosure, according to some implementations of the present disclosure. The illustrated computer 602 is intended to encompass any computing device such as a server, a desktop computer, a laptop/notebook computer, a wireless data port, a smart phone, a personal data assistant (PDA), a tablet computing device, or one or more processors within these devices, including physical instances, virtual instances, or both. The computer 602 can include input devices such as keypads, keyboards, and touch screens that can accept user information. Also, the computer 602 can include output devices that can convey information associated with the operation of the computer 602. The information can include digital data, visual data, audio information, or a combination of information. The information can be presented in a graphical user interface (UI) (or GUI).

The computer 602 can serve in a role as a client, a network component, a server, a database, a persistency, or components of a computer system for performing the subject matter described in the present disclosure. The illustrated computer 602 is communicably coupled with a network 630. In some implementations, one or more components of the computer 602 can be configured to operate within different environments, including cloud-computing-based environments, local environments, global environments, and combinations of environments.

At a top level, the computer 602 is an electronic computing device operable to receive, transmit, process, store, and manage data and information associated with the described subject matter. According to some implementations, the computer 602 can also include, or be communicably coupled with, an application server, an email server, a web server, a caching server, a streaming data server, or a combination of servers.

The computer 602 can receive requests over network 630 from a client application (for example, executing on another computer 602). The computer 602 can respond to the received requests by processing the received requests using software applications. Requests can also be sent to the computer 602 from internal users (for example, from a command console), external (or third) parties, automated applications, entities, individuals, systems, and computers.

Each of the components of the computer 602 can communicate using a system bus 603. In some implementations, any or all of the components of the computer 602, including hardware or software components, can interface with each other or the interface 604 (or a combination of both) over the system bus 603. Interfaces can use an application programming interface (API) 612, a service layer 613, or a combination of the API 612 and service layer 613. The API 612 can include specifications for routines, data structures, and object classes. The API 612 can be either computer-language independent or dependent. The API 612 can refer to a complete interface, a single function, or a set of APIs.

The service layer 613 can provide software services to the computer 602 and other components (whether illustrated or not) that are communicably coupled to the computer 602. The functionality of the computer 602 can be accessible for all service consumers using this service layer. Software services, such as those provided by the service layer 613, can provide reusable, defined functionalities through a defined interface. For example, the interface can be software written in JAVA, C++, or a language providing data in extensible markup language (XML) format. While illustrated as an integrated component of the computer 602, in alternative implementations, the API 612 or the service layer 613 can be stand-alone components in relation to other components of the computer 602 and other components communicably coupled to the computer 602. Moreover, any or all parts of the API 612 or the service layer 613 can be implemented as child or sub-modules of another software module, enterprise application, or hardware module without departing from the scope of the present disclosure.

The computer 602 includes an interface 604. Although illustrated as a single interface 604 in FIG. 6, two or more interfaces 604 can be used according to particular needs, desires, or particular implementations of the computer 602 and the described functionality. The interface 604 can be used by the computer 602 for communicating with other systems that are connected to the network 630 (whether illustrated or not) in a distributed environment. Generally, the interface 604 can include, or be implemented using, logic encoded in software or hardware (or a combination of software and hardware) operable to communicate with the network 630. More specifically, the interface 604 can include software supporting one or more communication protocols associated with communications. As such, the network 630 or the interface's hardware can be operable to communicate physical signals within and outside of the illustrated computer 602.

The computer 602 includes a processor 605. Although illustrated as a single processor 605 in FIG. 6, two or more processors 605 can be used according to particular needs, desires, or particular implementations of the computer 602 and the described functionality. Generally, the processor 605 can execute instructions and can manipulate data to perform the operations of the computer 602, including operations using algorithms, methods, functions, processes, flows, and procedures as described in the present disclosure.

The computer 602 also includes a database 606 that can hold data for the computer 602 and other components connected to the network 630 (whether illustrated or not). For example, database 606 can be an in-memory, conventional, or a database storing data consistent with the present disclosure. In some implementations, database 606 can be a combination of two or more different database types (for example, hybrid in-memory and conventional databases) according to particular needs, desires, or particular implementations of the computer 602 and the described functionality. Although illustrated as a single database 606 in FIG. 6, two or more databases (of the same, different, or combination of types) can be used according to particular needs, desires, or particular implementations of the computer 602 and the described functionality. While database 606 is illustrated as an internal component of the computer 602, in alternative implementations, database 606 can be external to the computer 602.

The computer 602 also includes a memory 607 that can hold data for the computer 602 or a combination of components connected to the network 630 (whether illustrated or not). Memory 607 can store any data consistent with the present disclosure. In some implementations, memory 607 can be a combination of two or more different types of memory (for example, a combination of semiconductor and magnetic storage) according to particular needs, desires, or particular implementations of the computer 602 and the described functionality. Although illustrated as a single memory 607 in FIG. 6, two or more memories 607 (of the same, different, or combination of types) can be used according to particular needs, desires, or particular implementations of the computer 602 and the described functionality. While memory 607 is illustrated as an internal component of the computer 602, in alternative implementations, memory 607 can be external to the computer 602.

The application 608 can be an algorithmic software engine providing functionality according to particular needs, desires, or particular implementations of the computer 602 and the described functionality. For example, application 608 can serve as one or more components, modules, or applications. Further, although illustrated as a single application 608, the application 608 can be implemented as multiple applications 608 on the computer 602. In addition, although illustrated as internal to the computer 602, in alternative implementations, the application 608 can be external to the computer 602.

The computer 602 can also include a power supply 614. The power supply 614 can include a rechargeable or non-rechargeable battery that can be configured to be either user- or non-user-replaceable. In some implementations, the power supply 614 can include power-conversion and management circuits, including recharging, standby, and power management functionalities. In some implementations, the power-supply 614 can include a power plug to allow the computer 602 to be plugged into a wall socket or a power source to, for example, power the computer 602, or recharge a rechargeable battery.

There can be any number of computers 602 associated with, or external to, a computer system containing computer 602, with each computer 602 communicating over network 630. Further, the terms “client,” “user,” and other appropriate terminology can be used interchangeably, as appropriate, without departing from the scope of the present disclosure. Moreover, the present disclosure contemplates that many users can use one computer 602 and one user can use multiple computers 602.

Described implementations of the subject matter can include one or more features, alone or in combination.

For example, in a first implementation, a computer-implemented method includes the following, receiving a collection of process parameters associated with disposal of water from a hydrocarbon extraction process, the collection of process parameters including a demulsifier flowrate, a hydrocarbon temperature, a first high pressure production trap (HPPT) interface level, a second HPPT interface level, a produced water rate, a dehydrator interface level, a desalter interface level, a tie line production rate, a water-oil separator oil level, and a water-in-oil separator water level, determining an oil-in-water (OIW) concentration in hydrocarbons from the hydrocarbon extraction process using the collection of parameters according to a first order linear regression model, comparing the determined OIW concentration to a threshold concentration, and adjusting at least one of the collection of parameters associated with the hydrocarbon extraction process based on the comparing.

The foregoing and other described implementations can each, optionally, include one or more of the following features:

A first feature, combinable with any of the following features, where the computer-implemented method includes providing a notification based on the comparing.

A second feature, combinable with any of the previous or following features, where the computer-implemented method includes receiving a sample of hydrocarbons output from the hydrocarbon extraction process, and comparing the determined OIW concentration to a OIW concentration determined from the received sample.

A third feature, combinable with any of the previous or following features, where determining the OIW concentration in hydrocarbons from the hydrocarbon extraction process using the collection of process parameters can include determining the OIW concentration according to Equation 1.

A fourth feature, combinable with any of the previous features, where receiving a collection of process parameters can further include periodically receiving the collection of parameters, and where determining an OIW concentration can further include periodically determining the OIW concentration.

In a second implementation, a non-transitory, computer-readable medium stores one or more instructions executable by a computer system to perform operations including receiving a collection of process parameters associated with disposal of water from a hydrocarbon extraction process, the collection of process parameters including a demulsifier flowrate, a hydrocarbon temperature, a first high pressure production trap (HPPT) interface level, a second HPPT interface level, a produced water rate, a dehydrator interface level, a desalter interface level, a tie line production rate, a water-oil separator oil level, and a water-in-oil separator water level, determining an oil-in-water (OIW) concentration in hydrocarbons from the hydrocarbon extraction process using the plurality of parameters according to a first order linear regression model, comparing the determined OIW concentration to a threshold concentration, and adjusting at least one of the collection of parameters associated with the hydrocarbon extraction process based on the comparing.

The foregoing and other described implementations can each, optionally, include one or more of the following features:

A first feature, combinable with any of the following features, further including providing a notification based on the comparing.

A second feature, combinable with any of the previous or following features, further including receiving a sample of hydrocarbons output from the hydrocarbon extraction process, and comparing the determined OIW concentration to an OIW concentration determined from the received sample.

A third feature, combinable with any of the previous or following features, where determining the OIW concentration in hydrocarbons from the hydrocarbon extraction process using the collection of process parameters can include determining the OIW concentration according to Equation 1.

A fourth feature, combinable with any of the previous features, where receiving a collection of process parameters can further include periodically receiving the collection of parameters, and where determining an OIW concentration can further include periodically determining the OIW concentration.

In a third implementation, a computer-implemented system includes one or more processors, and a non-transitory computer-readable storage medium coupled to the one or more processors and storing programming instructions for execution by the one or more processors, the programming instructions instructing the one or more processors to perform operations including receiving a collection of process parameters associated with disposal of water from a hydrocarbon extraction process, the collection of process parameters including a demulsifier flowrate, a hydrocarbon temperature, a first high pressure production trap (HPPT) interface level, a second HPPT interface level, a produced water rate, a dehydrator interface level, a desalter interface level, a tie line production rate, a water-oil separator oil level, and a water-in-oil separator water level, determining an oil-in-water (OIW) concentration in hydrocarbons from the hydrocarbon extraction process using the collection of parameters according to a first order linear regression model, comparing the determined OIW concentration to a threshold concentration, and adjusting at least one of the collection of parameters associated with the hydrocarbon extraction process based on the comparing.

The foregoing and other described implementations can each, optionally, include one or more of the following features:

A first feature, combinable with any of the following features, further including providing a notification based on the comparing.

A third feature, combinable with any of the previous or following features, further including receiving a sample of hydrocarbons output from the hydrocarbon extraction process, and comparing the determined OIW concentration to an OIW concentration determined from the received sample.

A third feature, combinable with any of the previous or following features, wherein determining the OIW concentration in hydrocarbons from the hydrocarbon extraction process using the collection of process parameters can include determining the OIW concentration according to Equation 1.

A fourth feature, combinable with any of the previous, where receiving a collection of process parameters can further include periodically receiving the collection of parameters, and where determining an OIW concentration can further include periodically determining the OIW concentration.

Although a few implementations have been described in detail above, other modifications are possible. For example, the logic flows depicted in the figures do not require the particular order shown, or sequential order, to achieve desirable results. In addition, other steps may be provided, or steps may be eliminated, from the described flows, and other components may be added to, or removed from, the described systems. Accordingly, other implementations are within the scope of the following claims.

What is claimed is:

1. A computer-implemented method, comprising:
receiving a plurality of process parameters associated with disposal of water from a hydrocarbon extraction process, the plurality of process parameters comprising a demulsifier flowrate, a hydrocarbon temperature, a first high pressure production trap (HPPT) interface level, a second HPPT interface level, a produced water rate, a dehydrator interface level, a desalter interface level, a tie line production rate, a water-oil separator oil level, and a water-in-oil separator water level;
determining an oil-in-water (OIW) concentration in hydrocarbons from the hydrocarbon extraction process using the plurality of process parameters according to a first order linear regression model;
comparing the determined OIW concentration to a threshold concentration; and
adjusting at least one of the plurality of process parameters associated with the hydrocarbon extraction process based on the comparing.

2. The method of claim 1, further comprising providing a notification based on the comparing.

3. The method of claim 1, further comprising:
receiving a sample of hydrocarbons output from the hydrocarbon extraction process; and
comparing the determined OIW concentration to an OIW concentration determined from the received sample.

4. The method of claim 1, wherein determining the OIW concentration in hydrocarbons from the hydrocarbon extraction process using the plurality of process parameters comprises determining the OIW concentration according to the following:
OIW=β₀+β₁A+β₂B+β₃C+β₄D+β₅E+β₆F+β₇G+β₈H+β₉I+β₁₀J+ε
wherein:
OIW is the oil-in-water concentration;
A is the demulsifier flowrate;
B is the hydrocarbon temperature;
C is the first HPPT interface level;
D is the second HPPT interface level;
E is the produced water rate;
F is the dehydrator interface level;
G is the desalter interface level;
H is the tie line production rate;
I is the water-oil separator oil level;
J is the water-in-oil separator water level;
ε is a random error term;
and β₀, β₁, β₂, β₃, β₄, β₅, and β₆ are factor effects.

5. The method of claim 1, wherein receiving a plurality of process parameters further comprises periodically receiving the plurality of process parameters, and wherein determining an OIW concentration further comprises periodically determining the OIW concentration.

6. A non-transitory, computer-readable medium storing one or more instructions executable by a computer system to perform operations comprising:
receiving a plurality of process parameters associated with disposal of water from a hydrocarbon extraction process, the plurality of process parameters comprising a demulsifier flowrate, a hydrocarbon temperature, a first high pressure production trap (HPPT) interface level, a second HPPT interface level, a produced water rate, a dehydrator interface level, a desalter interface level, a tie line production rate, a water-oil separator oil level, and a water-in-oil separator water level;
determining an oil-in-water (OIW) concentration in hydrocarbons from the hydrocarbon extraction process using the plurality of process parameters according to a first order linear regression model;
comparing the determined OIW concentration to a threshold concentration; and
adjusting at least one of the plurality of process parameters associated with the hydrocarbon extraction process based on the comparing.

7. The non-transitory, computer-readable medium of claim 6, wherein the operations further comprise providing a notification based on the comparing.

8. The non-transitory, computer-readable medium of claim 6, wherein the operations further comprise:
receiving a sample of hydrocarbons output from the hydrocarbon extraction process; and
comparing the determined OIW concentration to an OIW concentration determined from the received sample.

9. The non-transitory, computer-readable medium of claim 6, wherein determining the OIW concentration in hydrocarbons from the hydrocarbon extraction process using the plurality of process parameters comprises determining the OIW concentration according to the following:
OIW=β₀+β₁A+β₂B+β₃C+β₄D+β₅E+β₈F+β₇G+β₈H+β₉I+β₁₀J+ε
wherein:
OIW is the oil-in-water concentration;
A is the demulsifier flowrate;
B is the hydrocarbon temperature;
C is the first HPPT interface level;
D is the second HPPT interface level;
E is the produced water rate;
F is the first dehydrator interface level;
G is the second dehydrator interface level;
H is the tie line production rate;
I is the water-oil separator oil level;
J is the water-in-oil separator water level;
E is a random error term;
and β₀, β₁, β₂, β₃, β₄, β₅, and β₆ are factor effects.

10. The non-transitory, computer-readable medium of claim 6, wherein receiving a plurality of process parameters further comprises periodically receiving the plurality of process parameters, and wherein determining an OIW concentration further comprises periodically determining the OIW concentration.

11. A computer-implemented system, comprising:
one or more processors; and
a non-transitory computer-readable storage medium coupled to the one or more processors and storing programming instructions for execution by the one or more processors, the programming instructions instructing the one or more processors to perform operations comprising:
receiving a plurality of process parameters associated with disposal of water from a hydrocarbon extraction process, the plurality of process parameters comprising a demulsifier flowrate, a hydrocarbon temperature, a first high pressure production trap (HPPT) interface level, a second HPPT interface level, a produced water rate, a dehydrator interface level, a desalter interface level, a tie line production rate, a water-oil separator oil level, and a water-in-oil separator water level;
determining an oil-in-water (OIW) concentration in hydrocarbons from the hydrocarbon extraction process using the plurality of process parameters according to a first order linear regression model;
comparing the determined OIW concentration to a threshold concentration; and
adjusting at least one of the plurality of process parameters associated with the hydrocarbon extraction process based on the comparing.

12. The computer-implemented system of claim 11, wherein the operations further comprise providing a notification based on the comparing.

13. The computer-implemented system of claim 11, further comprising:
receiving a sample of hydrocarbons output from the hydrocarbon extraction process; and
comparing the determined OIW concentration to an OIW concentration determined from the received sample.

14. The computer-implemented system of claim 11, wherein determining the OIW concentration in hydrocarbons from the hydrocarbon extraction process using the plurality of process parameters comprises determining the OIW concentration according to the following:
OIW=β₀+β₁A+β₂B+β₃C+β₄D+β₅E+β₈F+β₇G+β₈H+β₉I+β₁₀J+ε
wherein:
OIW is the oil-in-water concentration;
A is the demulsifier flowrate;
B is the hydrocarbon temperature;
C is the first HPPT interface level;
D is the second HPPT interface level;
E is the produced water rate;
F is the first dehydrator interface level;
G is the second dehydrator interface level;
H is the tie line production rate;
I is the water-oil separator oil level;
J is the water-in-oil separator water level;
ε is a random error term;
and β₀, β₁, β₂, β₃, β₄, β₅, and β₆ are factor effects.

15. The computer-implemented system of claim 11, wherein receiving a plurality of process parameters further comprises periodically receiving the plurality of process parameters, and wherein determining an OIW concentration further comprises periodically determining the OIW concentration.
